(19) United States
(12) Patent Application Publication
(10) Pub. No.: US 2013/0289512 A1
(43) Pub. Date: Oct. 31, 2013

(54) Disposable Garment Having Soft, Discreet Seams

(71) Applicants: Brian Keith Rhodes, Larsen, WI (US); Lacey Leigh Poole, Appleton, WI (US); Marlene Ruth Dins, Appleton, WI (US); Joseph Andrew Mlinar, Appleton, WI (US); Paul Douglas Beuther, Neenah, WI (US); Marie Elizabeth Boll, Appleton, WI (US); Paulin Pawar, Appleton, WI (US)

(72) Inventors: Brian Keith Rhodes, Larsen, WI (US); Lacey Leigh Poole, Appleton, WI (US); Marlene Ruth Dins, Appleton, WI (US); Joseph Andrew Mlinar, Appleton, WI (US); Paul Douglas Beuther, Neenah, WI (US); Marie Elizabeth Boll, Appleton, WI (US); Paulin Pawar, Appleton, WI (US)

(21) Appl. No.: 13/460,311

(22) Filed: Apr. 30, 2012

(51) Int. Cl. A61F 13/493; A41D 1/06; A41D 1/00

(52) U.S. Cl. 604/385.24; 2/69; 2/227

(57) ABSTRACT

A disposable garment having a fin seam, wherein the fin seam has a Height no greater than 6 millimeters and a Rigidity no greater than 0.6 gram-force/%-compression as measured by the Test Method. In particular embodiments, the garment is a pant-like garment having two outwardly extending side fin seams, each having a Height no greater than 6 millimeters and a Rigidity no greater than 0.6 gram-force/%-compression as measured by the Test Method.

### BACKGROUND

Disposable garments, such as disposable absorbent pant-like garments, are in widespread use in society. For example, disposable incontinence underwear, disposable enuresis underwear, disposable toilet training pants, and disposable pull-on diapers are common. In one common embodiment of such garments, a front region and a back region are interconnected by a crotch region, and the front region and the back region are further connected to each other along a pair of side seams, such that the garment assumes a pull-on style form having a waist opening and two leg openings. In addition to the ability to provide protection against leakage, it is desirable for such products to provide both comfort and discretion to the wearer.

Unfortunately, the seams of such products, such as the side seams described above, have not delivered acceptable levels of softness and discretion. The inventors have found that by making the side seam as narrow as possible, the degree of discretion offered by the garment is maximized. Furthermore, the inventors have determined that by making the side seam as soft and flexible as possible, the degree of comfort offered to the wearer is maximized

Strohbeen et al. recognized the desirability of providing an exterior seam or seal on a disposable garment that is narrow and therefore discreet in U.S. Pat. No. 4,610,681, assigned to Kimberly-Clark Worldwide, Inc. For example, Strohbeen et al. teaches the desirability of a side seam having a width of between 1/16″ and 3/16″ inches, and preferably having a width of ⅛″. However, Strohbeen offers no teaching on how to construct a narrow side seam so as to be soft and flexible. Furthermore, after extensive testing of commercially available disposable garments having bonded side seams, the present inventors have been unsuccessful in finding garments whose side seams are both sufficiently narrow and sufficiently soft/flexible.

Accordingly, what is needed is a disposable garment having seams that are both narrow/discreet, as well as soft/flexible.

### SUMMARY OF THE INVENTION

In one embodiment, the present invention relates to a disposable garment having a fin seam, wherein the fin seam has a Height no greater than 6 millimeters and a Rigidity no greater than 0.6 gram-force/%-compression as measured by the Test Method.

In another embodiment, the present invention relates to a disposable, pant-like garment having a waist opening, first and second leg openings, a first side fin seam extending substantially from the waist opening to the first leg opening, and a second side fin seam extending substantially from the waist opening to the second leg opening. Each side fin seam has a Height no greater than 6 millimeters and a Rigidity no greater than 0.6 gram-force/%-compression as measured by the Test Method.

In yet another embodiment, the present invention relates to a disposable, pant-like, absorbent garment defining a longitudinal direction and a transverse direction, a front region, a back region, and a crotch region, the garment having a front center panel and a back center panel, the front region including first and second elastomeric front side panels extending transversely from the front center panel, the back region including first and second elastomeric back side panels extending transversely from the back center panel. The first elastomeric front side panel is bonded to the first elastomeric back side panel along an outwardly extending first side fin seam, and the second elastomeric front side panel is bonded to the second elastomeric back side panel along an outwardly extending second side fin seam. Each side panel comprises an elastomeric film laminate. Each side fin seam has a Height no greater than 6 millimeters and a Rigidity no greater than 0.6 gram-force/%-compression as measured by the Test Method. The garment can include an absorbent composite.

### BRIEF DESCRIPTION OF THE DRAWINGS

FIG. 1 shows a top plan view of an embodiment of a garment suitable for use in conjunction with particular embodiments of the present invention, shown in a laid-open configuration prior to the creation of side seams, with the surface configured to contact the wearer's skin shown facing up.

FIG. 2 shows a front perspective view of the garment of FIG. 1 shown in a fully assembled configuration following creation of side seams.

FIG. 3 shows an alternative embodiment of the garment of FIG. 2 that also includes a pair of refastenable seams.

FIG. 4 shows a top plan view of an alternative embodiment of a garment suitable for use in conjunction with particular embodiments of the present invention, shown in a laid-open configuration prior to the creation of side seams, with the surface configured to contact the wearer's skin shown facing up.

FIG. 5 shows a perspective view of the garment of FIG. 4 shown in a fully assembled configuration following creation of side seams.

FIG. 6 shows a close-up plan view of a portion of a side seam of the garment depicted in FIG. 2.

FIG. 7 shows a cross-sectional view of the side seam of FIG. 6 taken along line 7-7.

FIG. 8 shows a front perspective view of a compression tester suitable for use in conjunction with the Test Method disclosed herein.

FIG. 9 shows a front perspective view of a modified version of the compression tester of FIG. 8.

FIG. 10 shows a close-up perspective view of a portion of the compression tester of FIG. 9.

FIG. 11 shows a modified view of the compression tester portion shown in FIG. 10.

FIGS. 12, 14, and 15 show representative perspective views of various stages of the Test Method disclosed herein.

FIG. 13 shows a cross-sectional view of a fin seam sample mounted on a sample mounting plate in accordance with the Test Method disclosed herein.

FIG. 16 shows a top plan view of a stage of the Test Method disclosed herein.

FIG. 17 representatively illustrates how to prepare a fin seam sample from a disposable garment in accordance with the Test Method disclosed herein.

FIG. 18 presents representative average compression curves for fin side seams present in both commercially available products and experimental embodiments of the present invention.

### DEFINITIONS

Within the context of this specification, each term or phrase below will include the following meaning or meanings. Additional terms are defined elsewhere in the specification.

“Bonded” refers to the joining, adhering, connecting, attaching, or the like, of two elements. Two elements will be considered to be bonded together when they are bonded directly to one another or indirectly to one another, such as when each is directly bonded to intermediate elements.

“Disposable” refers to articles which are designed to be discarded after a limited use rather than being laundered or otherwise restored for reuse.

“Elastic,” “elasticized” and “elasticity” mean that property of a material or composite by virtue of which it tends to recover its original size and shape after removal of a force causing a deformation.

“Elastomeric” refers to a material or composite which can be elongated by at least 50 percent of its relaxed length and which will recover, upon release of the applied force, at least 20 percent of its elongation. It is generally preferred that the elastomeric material or composite be capable of being elongated by at least 100 percent, more preferably by at least 200 percent, of its relaxed length and recover, upon release of an applied force, at least 50 percent of its elongation.

“Integral” is used to refer to various portions of a single unitary element rather than separate structures bonded to or placed with or placed near one another.

“Layer” when used in the singular can have the dual meaning of a single element or a plurality of elements.

“Longitudinal” and “transverse” have their customary meaning, as indicated by the longitudinal and transverse axes depicted in the Figures. The longitudinal axis lies in the plane of the article and is generally parallel to a vertical plane that bisects a standing wearer into left and right body halves when the article is worn. The transverse axis lies in the plane of the article generally perpendicular to the longitudinal axis.

These terms may be defined with additional language in the remaining portions of the specification.

### DETAILED DESCRIPTION OF EXEMPLARY EMBODIMENTS

Reference to the Figures shall be made in describing various aspects and embodiments of the invention. It should be noted that the embodiments depicted in the Figures and described herein are merely representative examples of the garments of the invention. The various embodiments of the present invention can take the form of, for example, adult incontinence pants, open or pull-on disposable diapers, disposable swim pants, disposable training pants, disposable enuresis garments, surgical gowns, disposable clothing, menstrual panties, and the like.

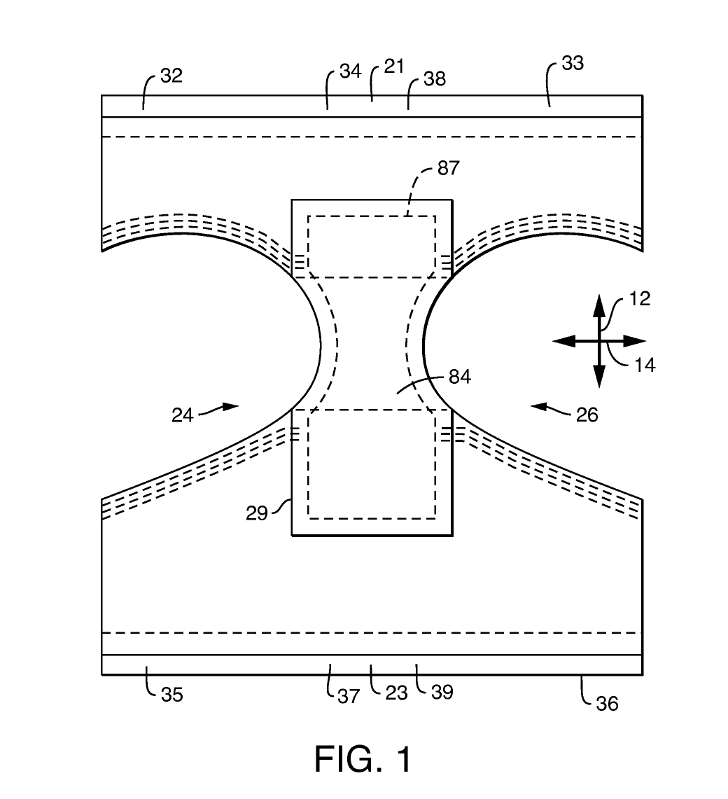

In particular embodiments, referring to FIGS. 1-3, the present invention relates to a disposable garment 20 having a fin seam. In particular embodiments, the garment defines a waist opening 22 and first and second leg openings 24, 26. The garment 20 includes a first side fin seam 30 extending at least part of the way from the waist opening 22 to the first leg opening 24, such as 100% of the way from the waist opening 22 to the first leg opening 24. The garment 20 includes a second side fin seam 31 extending at least part of the way from the waist opening 22 to the second leg opening 26, such as 100% of the way from the waist opening 22 to the second leg opening 26. “Fin seam” as used herein is a term understood by those in the art of disposable garments and refers to a flange formed when two panels of material are joined together in close proximity to an edge of each panel, the flange protruding from a primary surface of the garment. In particular embodiments, the referenced edges of the two panels are flush with each other.

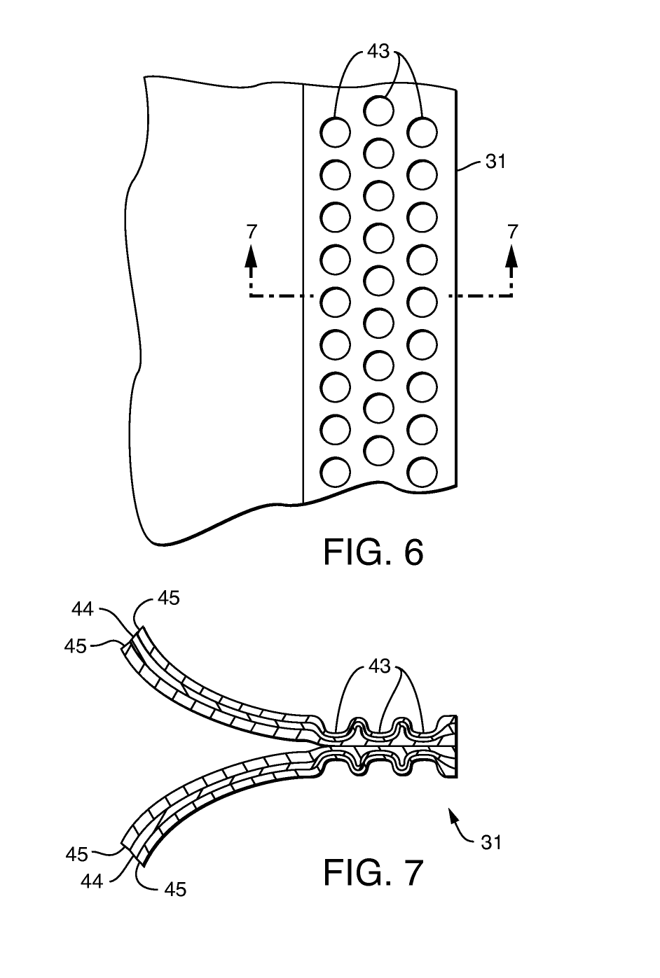

In one example, the garment is a disposable, pant-like, absorbent garment 20 that defines a longitudinal direction 12 and a transverse direction 14, a front region 21, a back region 23, and a crotch region 25. The front region 21 includes first and second front side panels 32, 33 extending transversely from a front center panel 34, and the back region 23 includes first and second back side panels 35, 36 extending transversely from a back center panel 37. In particular embodiments, the first front side panel 32 is bonded to the first back side panel 35 along a first side fin seam 30, and the second front side panel 33 is bonded to the second back side panel 36 along a second side fin seam 31. The fin seams 30, 31 can comprise a bond formed via ultrasonic, heat, or pressure energy, or can be formed via other suitable means. FIGS. 6 and 7 representatively illustrate a close-up view of a portion of a side seam suitable for use in conjunction with the present invention.

The front side panels 32, 33, the back side panels 35, 36, or preferably both the front side panels and the back side panels are elastomeric. In particular embodiments, each side panel comprises an elastomeric film 44, and in particular embodiments each side panel comprises an elastomeric film throughout each side fin seam 30, 31. Preferably, each side panel 32, 33, 35, 36 comprises an elastomeric film laminate. In particular embodiments, the front center panel 34, the back center panel 37, or both the front and back center panels 34, 37 comprise an elastomeric film, and preferably comprise an elastomeric film laminate.

Examples of elastomeric film laminates suitable for use in the side panels and the center panels are disclosed in U.S. Pat. No. 7,803,244 issued Sep. 28, 2010 to Siqueira et al. and assigned to Kimberly-Clark Worldwide, Inc., the entirely of which is hereby incorporated by reference to the extent not inconsistent herewith. In one example, the elastomeric film laminate employed in the side panels is elastomeric film 44 sandwiched between two nonwoven layers 45, 45 (FIG. 7). The elastomeric film layer is Vistamaxx 6102FL, having a basis weight of 30 grams per square meter in the relaxed state, available from ExxonMobil Corp., and each nonwoven facing layer comprises a spunbond nonwoven fabric made from SFT-315 polypropylene (available from ExxonMobil Corp.), each facing layer having a basis weight of 0.5 ounces per square yard in the fully extended state. The three layers are laminated together with heat bonds, and the composite laminate has a basis weight of 95 grams per square meter in the relaxed state.

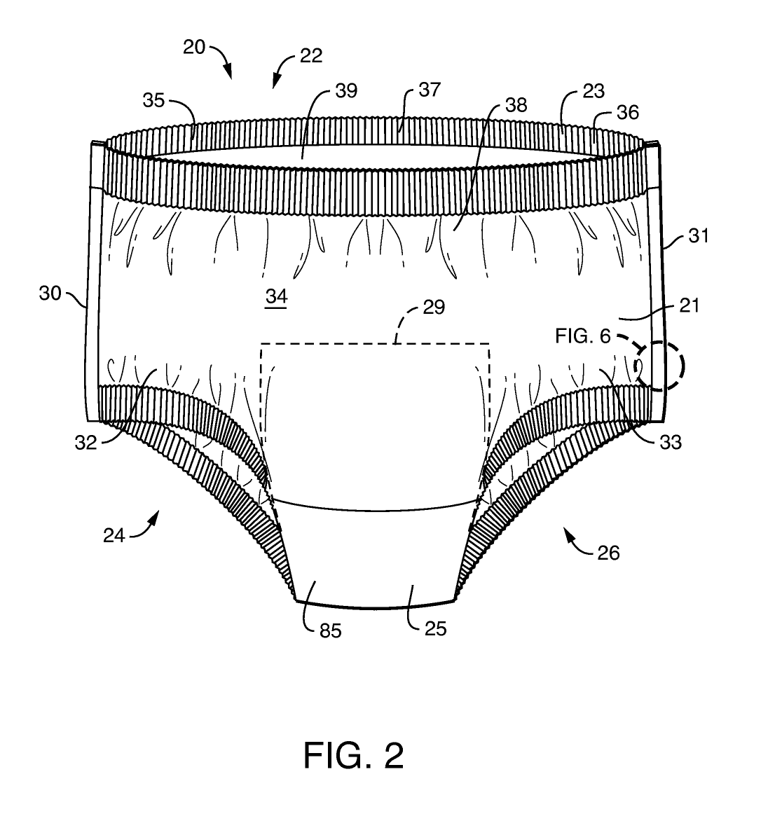

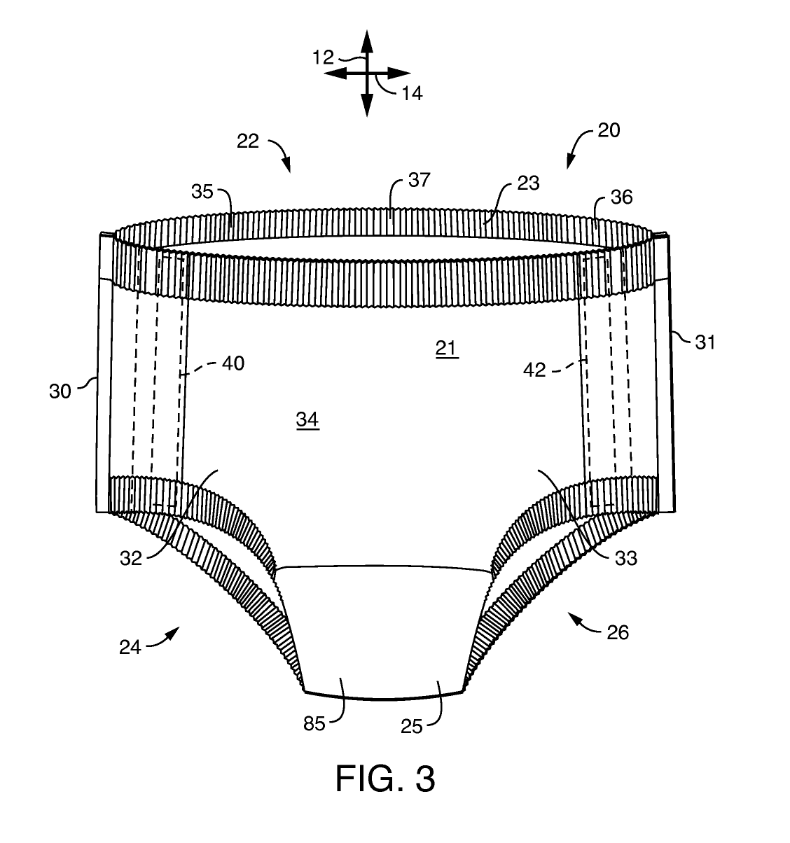

In particular embodiments, the front center panel 34, the first front side panel 32, and the second front side panel 33 together integrally form a unitary front body panel 38, representatively illustrated in FIGS. 1 and 2. Similarly, in particular embodiments, the back center panel 37, the first back side panel 35, and the second back side panel 36 together integrally form a unitary back body panel 39, representatively illustrated in FIGS. 1 and 2. In one preferable embodiment, a front body panel 38 and a back body panel 39 are each formed of an elastomeric film laminate. In one option, the garment includes one or more releasable, refastenable seams 40, 41, representatively illustrated in FIG. 3. The refastenable seams 40, 41 can in particular embodiments extend the majority, substantially the entirety, or the entirety of the distance from the waist opening 22 to the leg openings 24, 26.

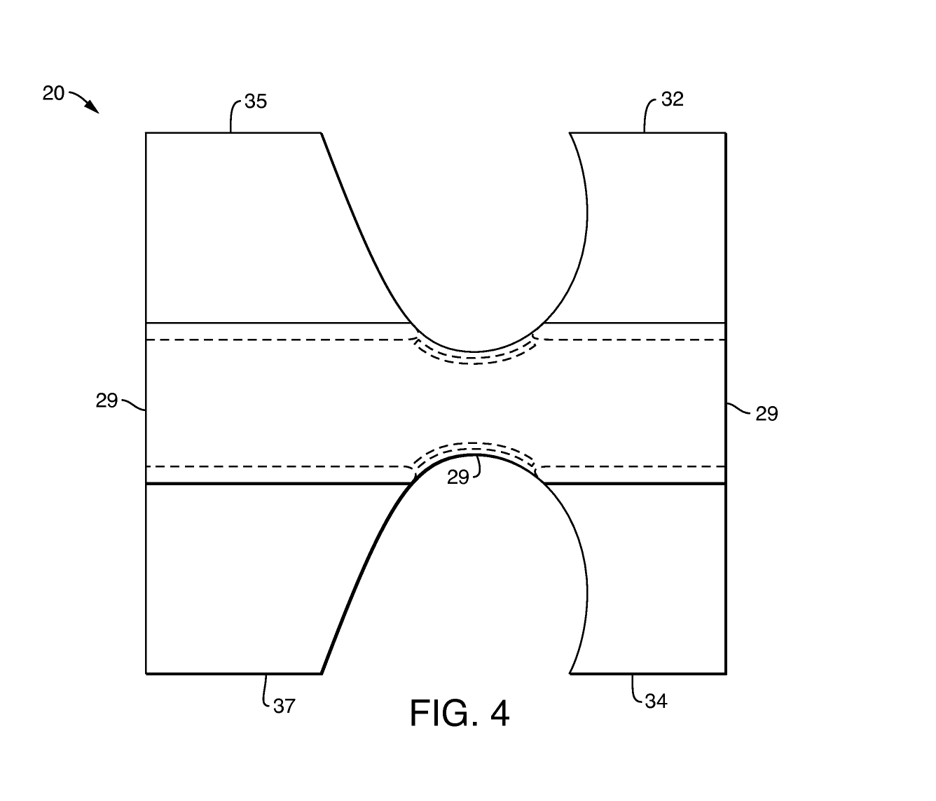

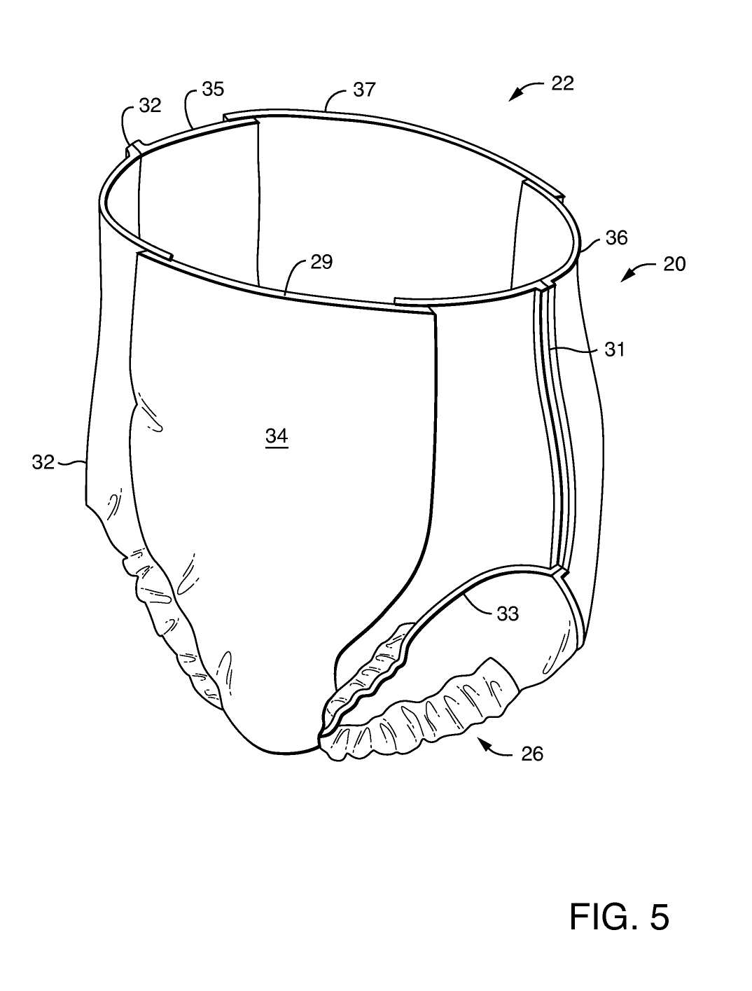

In particular embodiments, the garment 20 includes an absorbent composite 29. In particular embodiments, the absorbent composite comprises a liquid-permeable body-facing liner 84 configured to contact a wearer's skin, a liquid-impermeable backsheet 85 that faces away from the wearer's skin, and a fluid-absorbing core 87 sandwiched between the liner 84 and the backsheet 85. In one embodiment of the garment 20, the absorbent composite 29 does not extend to the waist opening, as representatively illustrated in FIGS. 1-3. In another embodiment of the garment 20, the absorbent composite 29 extends to the waist opening, as representatively illustrated in FIGS. 4 and 5.

In particular embodiments, one or more fin seams extend outward from the garment. “Extend outward” or “outwardly extending” as used herein means to protrude generally in a direction away from the wearer of the garment. For example, referring to FIGS. 2, 3, and 5, the side fin seams 30, 31 are seen to extend outward in the sense that the seams extend away from the body of the person donning the garment 20.

Each fin seam in the garment 20 has a height no greater than 6.0 millimeters, still more particularly no greater than 5.5 millimeters, and still more particularly no greater than 5.3 millimeters as measured by the Test Method described below. Additionally, each fin seam in the garment 20 has a rigidity no greater than 0.6, more particularly no greater than 0.5, still more particularly no greater than 0.4, still more particularly no greater than 0.3, and still more particularly no greater than 0.22 grams-force per %-compression, as measured by the Test Method described below.

### Test Method

The purpose of the following Test Method is to measure both the height and the rigidity of fin seams used in conjunction with the present invention. In summary, a probe attached to a load cell dynamically presses down on a fin seam, and both height and rigidity (resistance to compression) data is collected.

Equipment

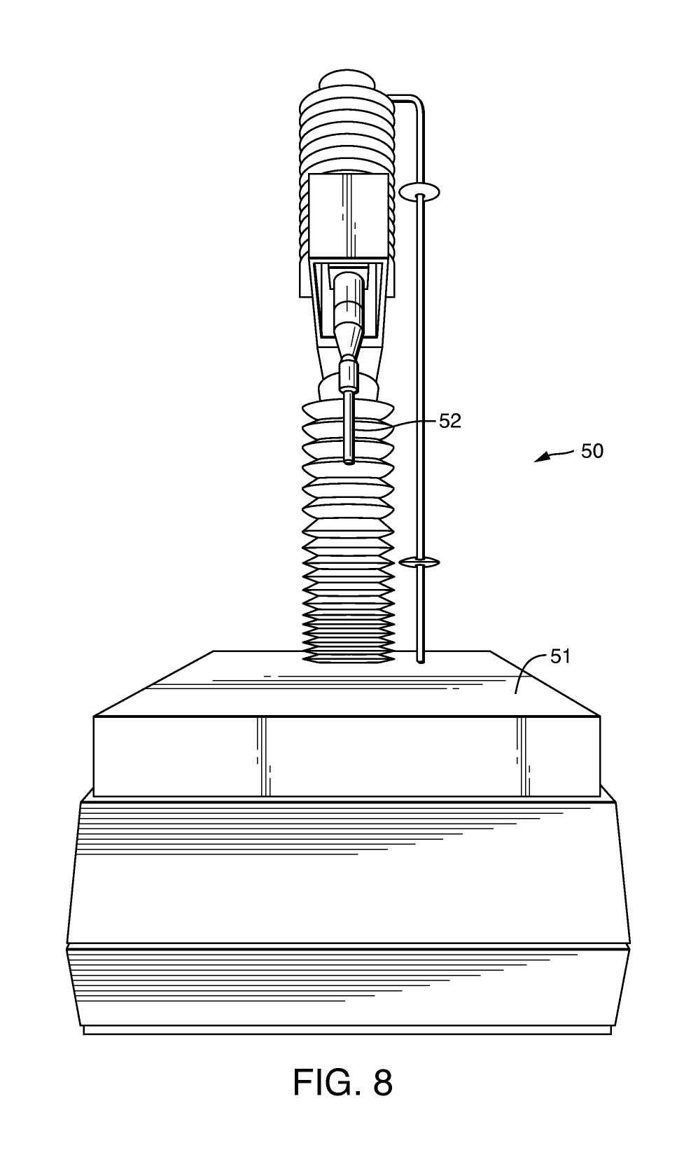

1. Compression tester 50—Texture Analyzer, model TA.XT Plus, manufactured by Texture Technologies Corp., 18 Fairview Road, Scarsdale, N.Y. (FIG. 8).

2. Data acquisition software—Exponent Stable Micro System Version 5,1,1,0., available from Texture Technologies Corp.

3. Cylinder platen 52, six (6) millimeters in diameter (part number P/6: 6 millimeter DIA CYLINDER STAINLESS, available from Texture Technologies Corp.), affixed to the load cell.

4. Two (2) kilogram calibration weight, available from Texture Technologies Corp.

5. Sample Mounting Plate 54—smooth aluminum or stainless steel plate, approximately 15 centimeters by 7.5 centimeters and 0.8 millimeters thick.

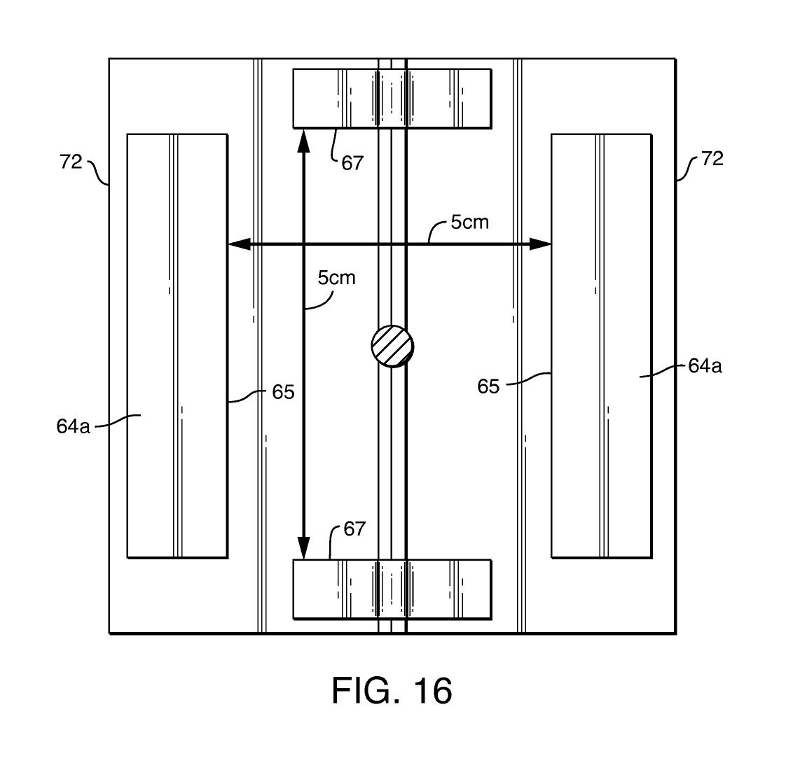

6. Lower Mounting Fixture 56. The Lower Mounting Fixture 56 utilized had a top pedestal section 58 approximately 25 centimeters long and 6 centimeters wide, having a top surface 60 held approximately 10 centimeters above the base 51 of the compression tester 50 by a support post 62. Two retaining clamps 64, 64 (such as horizontal hold-down toggle locking clamps, part no. 206-SS from DE-STA-CO, modified to include rectangular jaws 64a, 64a), are mounted to the top surface 60 of the pedestal section 58 such that their position is adjustable from one side of the compression tester to the other (such as via a Left-Right Screw Motion UniSlides Series A25 Model #2509C-S2.5-LR linear slide 69 from Velmex, Inc., Bloomfield, N.Y.). The inner edges 65, 65 of the clamp jaws 64a, 64a are spaced five (5) centimeters from each other when the clamps are in the closed position 68 (FIG. 16). Each clamp jaw 64a, 64a has a jaw footprint having a length of 50 millimeters and a width of 12 millimeters. The Lower Mounting Fixture 56 is configured such that the clamps 64, 64 can securely hold in place the sample 70 and the sample mounting plate 54, and each clamp jaw 64a, 64a includes a coating, such as hook- or mushroom-style mechanical fasteners, to prevent slippage of the sample mounting plate 54 and sample 70. The clamps 64, 64 are configured to pinch the sample 70 and the sample mounting plate 54 on opposite sides of the fin seam 30.

7. Masking tape—25 millimeters wide, model no. 234 from the 3M Company, St. Paul, Minn., or comparable equivalent (i.e., suitable for performing the function described below).

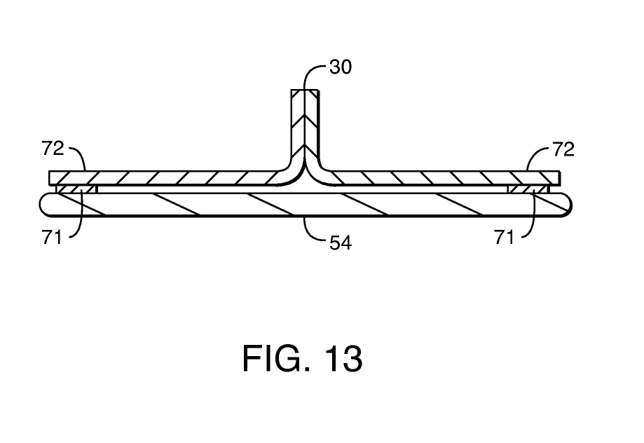

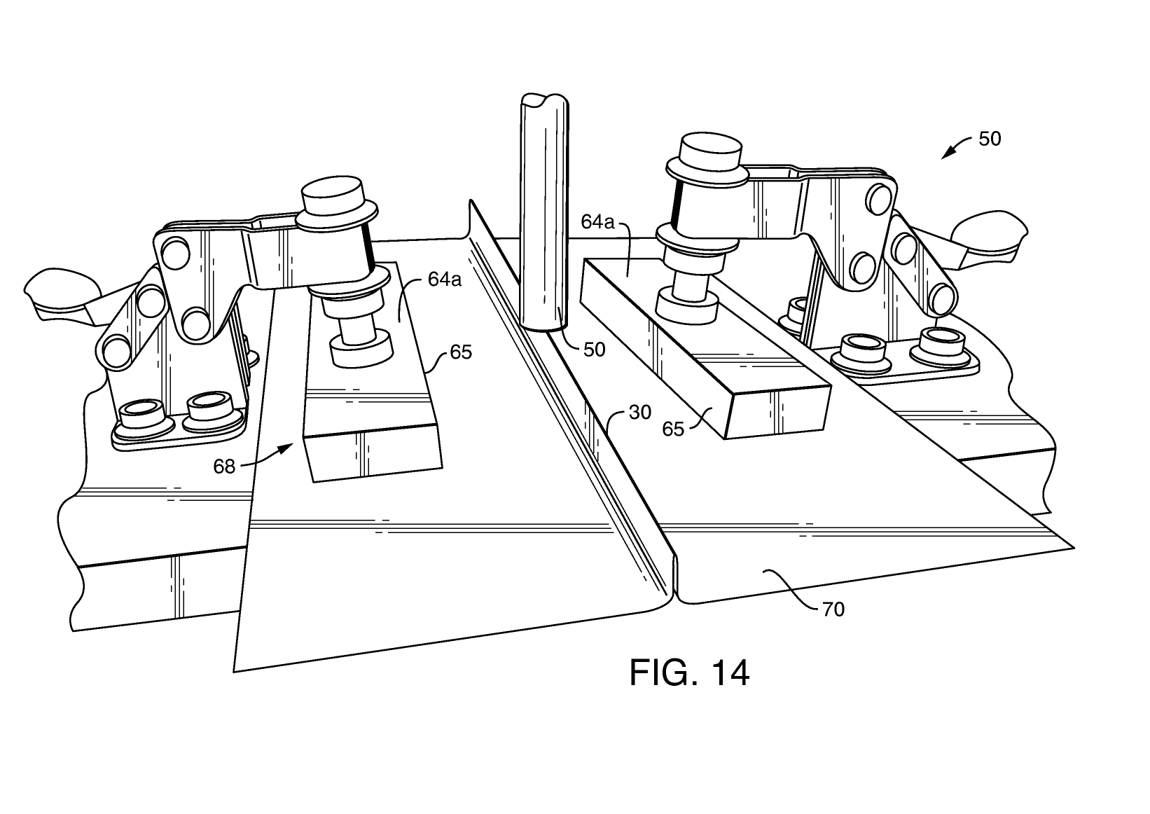

8. Hook- or mushroom-style mechanical fasteners 71 (FIG. 13), capable of engaging the sample 70.

Table 1 lists the settings that are employed in the Test Method. The data acquisition rate is set to 25 points per second. The load cell capacity is five (5) kilograms.

TABLE 1
Test Program Settings

| Texture Analyzer Settings | Selection |
|---|---|
| Test Mode | Compression |
| Pre-Test Speed | 0.5 millimeters/second |
| Test Speed | 0.5 millimeters/second |
| Post Test Speed | 5.0 millimeters/second |
| Target Mode | Force |
| Force | 10 grams |
| Trigger Type | Auto (Force) |
| Trigger Force | 0.5 grams |
| Break Mode | Off |
| Stop Plot at | Trigger Return |
| Tare Mode | Auto |

Test Procedure

1. Warm up the compression tester 50, and calibrate and verify the load cell force output according to the manufacturer's manual.

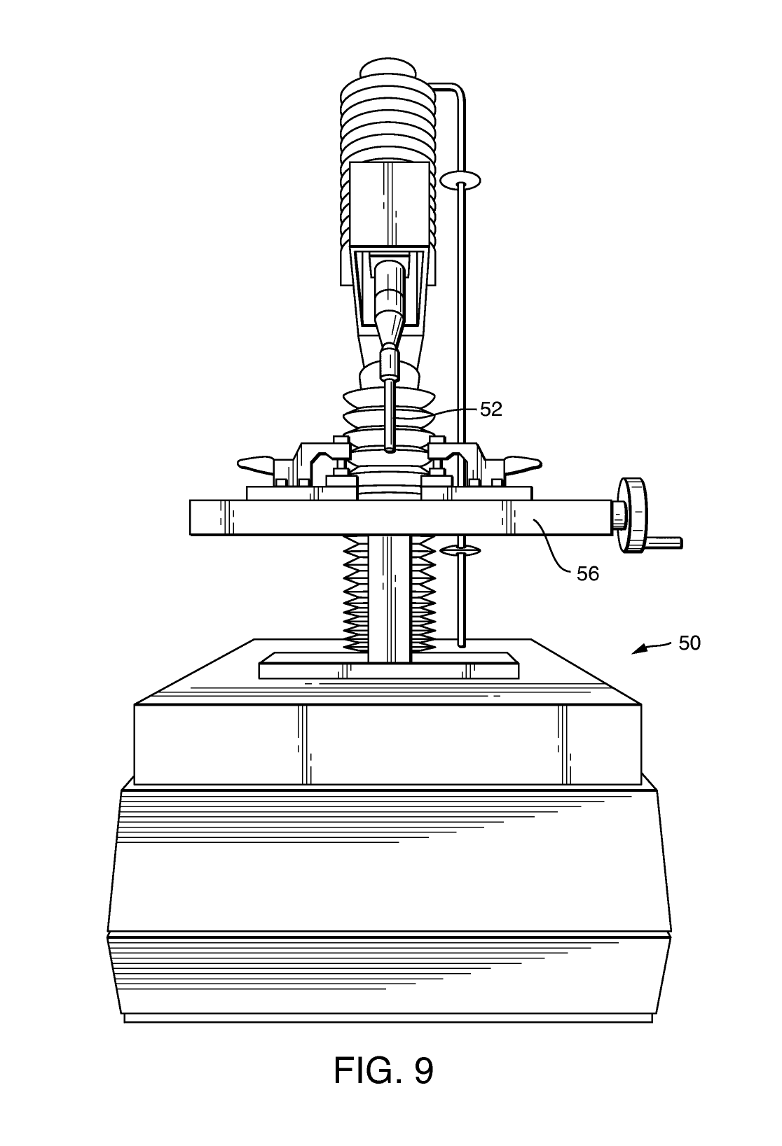

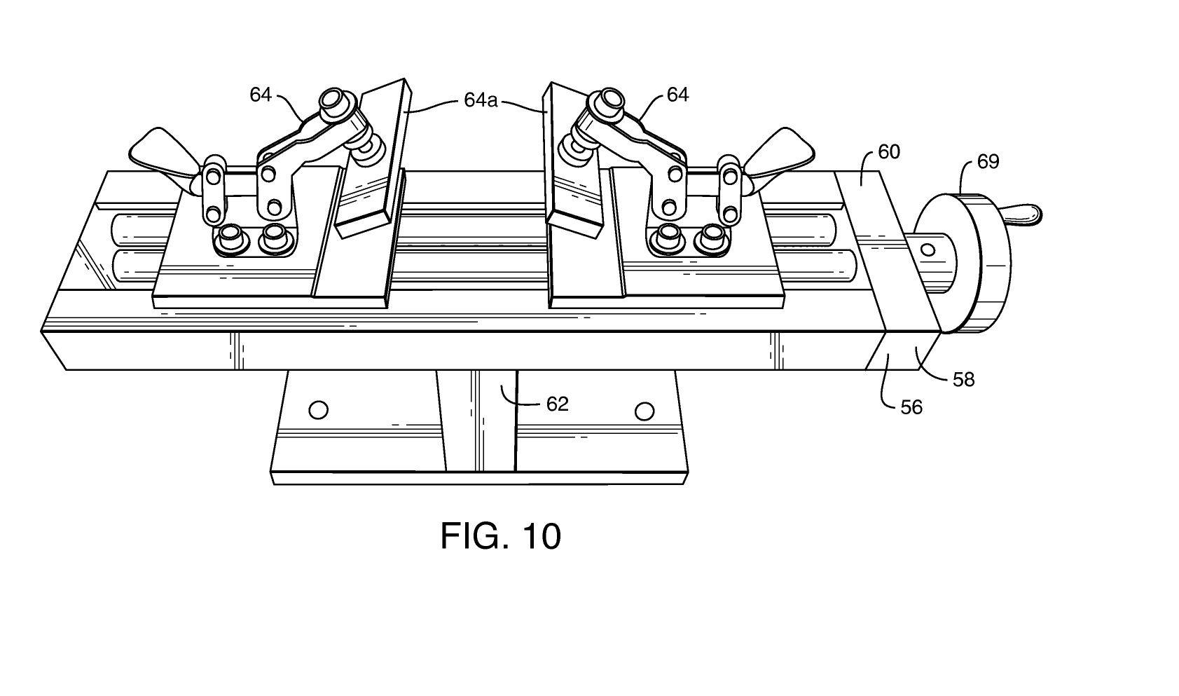

2. Position the cylinder platen 52 at the upper test arm and position the lower mounting fixture 56 as shown in FIGS. 8 and 9.

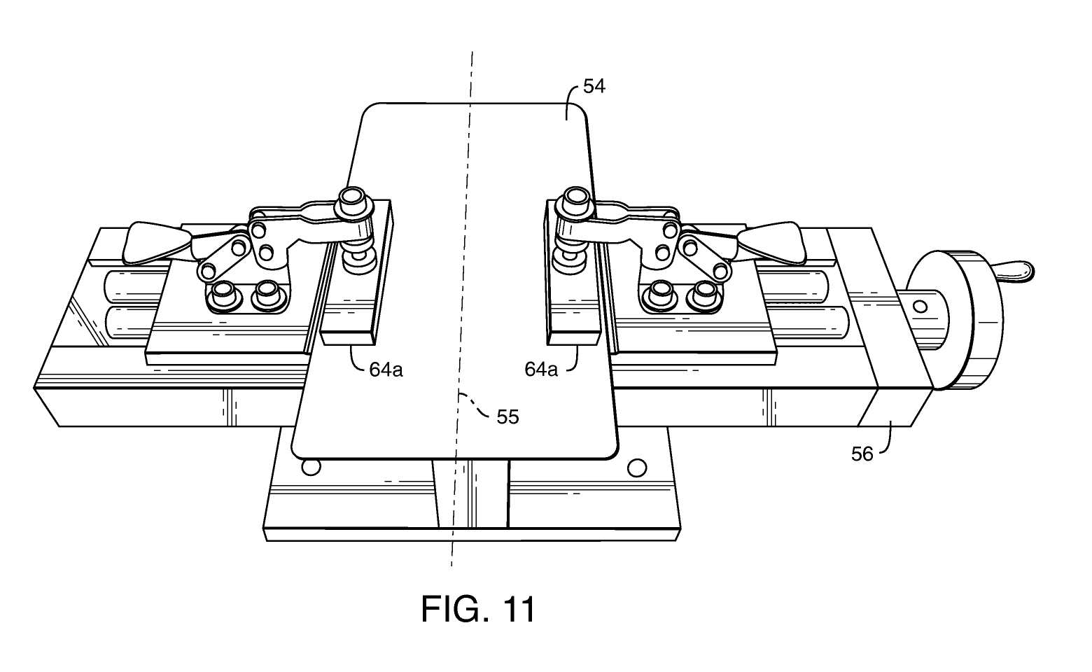

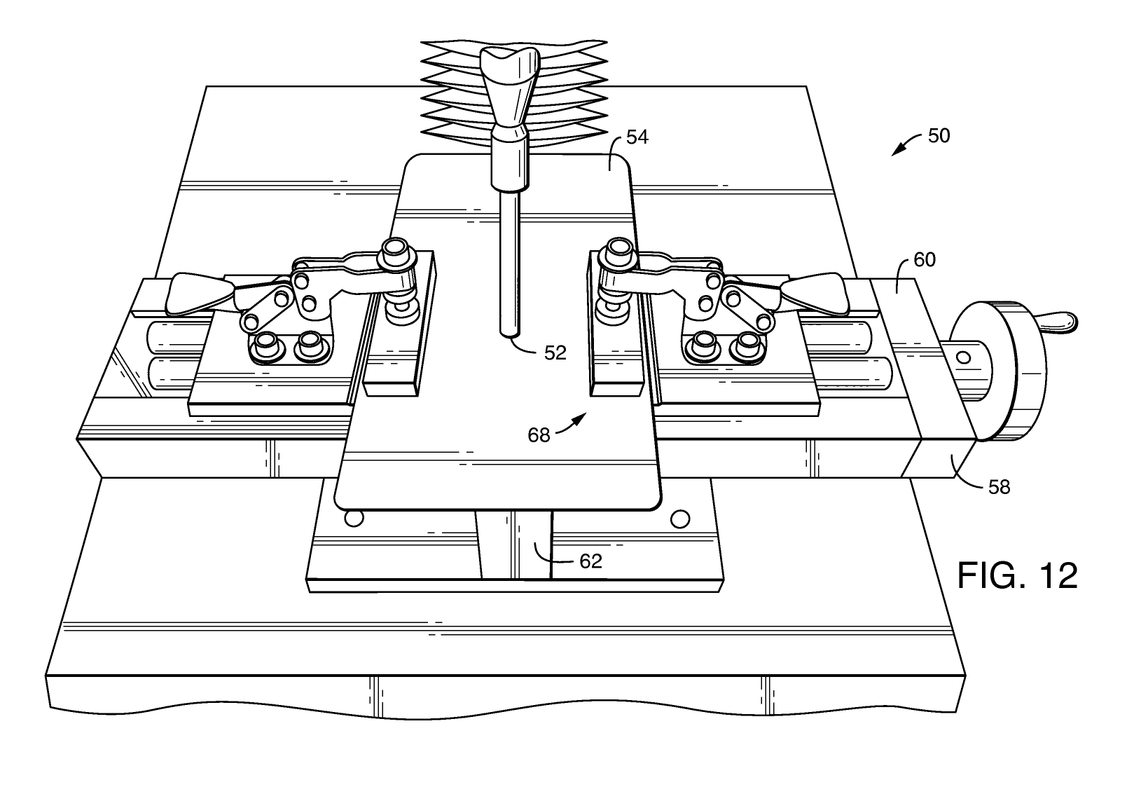

3. Clamp the mounting plate 54 within the clamps 64, 64 as shown in FIGS. 11 and 12 to establish a zero point and to provide a reference point to measure the displacement of the probe in the subsequent test runs. To set the initial position of the probe, click on “Probe Height Calibration” and set “Return Distance” at 15 mm, “Return Speed” at 5 mm/s and “Contact Force” at 5 g.

4. Set up the test program in the “T.A. Settings” in accordance with the settings set forth in Table 1.

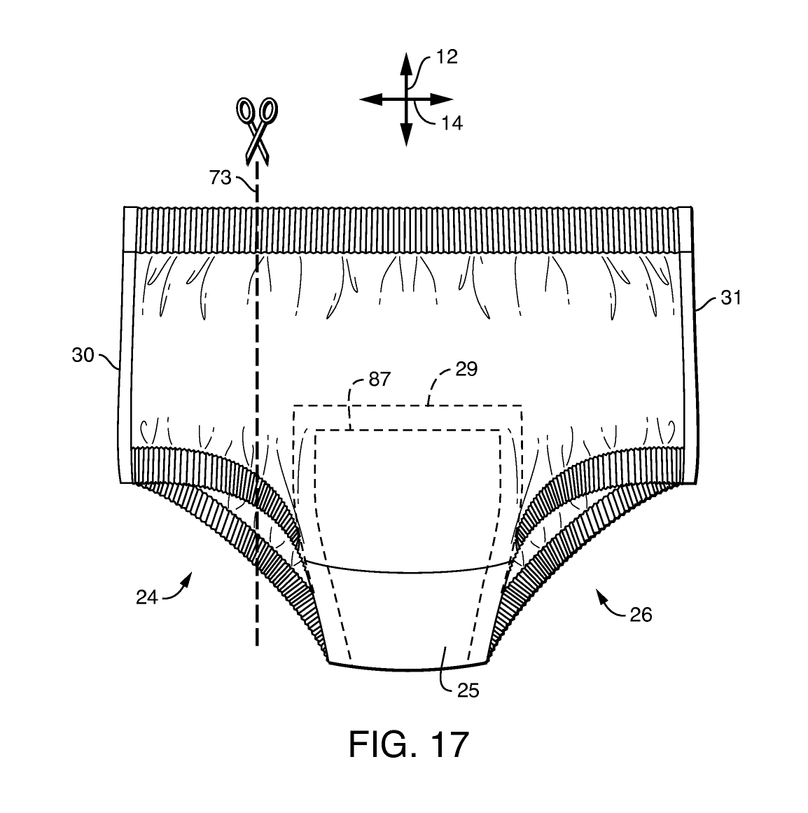

5. Isolate the fin seam portion of the garment by cutting along lines that run parallel to and that are spaced approximately four (4) centimeters from the fin seam. FIG. 17 shows a fin side seam being isolated from a pant-like disposable undergarment along a cut line 73. If the cut line 73 overlaps any portion of an absorbent core, scrape all absorbent core material from the sample 70 prior to positioning the sample on the test equipment. If the fin seam of the garment to be examined is longer than fifteen (15) centimeters, isolate a 15-centimeter long section of the fin seam, such that the fin seam portion to be examined does not exceed the length of the mounting plate 54.

6. Position the sample 70 atop the plate so that the fin seam extends along a longitudinal centerline 55 of the plate 54. If the sample 70 is elastomeric, stretch the sample slightly (i.e., up to approximately 15-20% elongation) to make the sample slightly taut. Secure the side portions 72, 72 of the sample 70 to the upward-facing surface of the mounting plate 54 using adhesive-backed mechanical fastener strips 71, 71 (such that the adhesive adheres the mechanical fastener strips to the plate 54 and such that the mechanical engaging elements engage the nonwoven sample 70), or other suitable securing means.

7. Clamp the mounting plate 54 along with the sample 70 to the lower mounting fixture 56 using the clamps 64, 64.

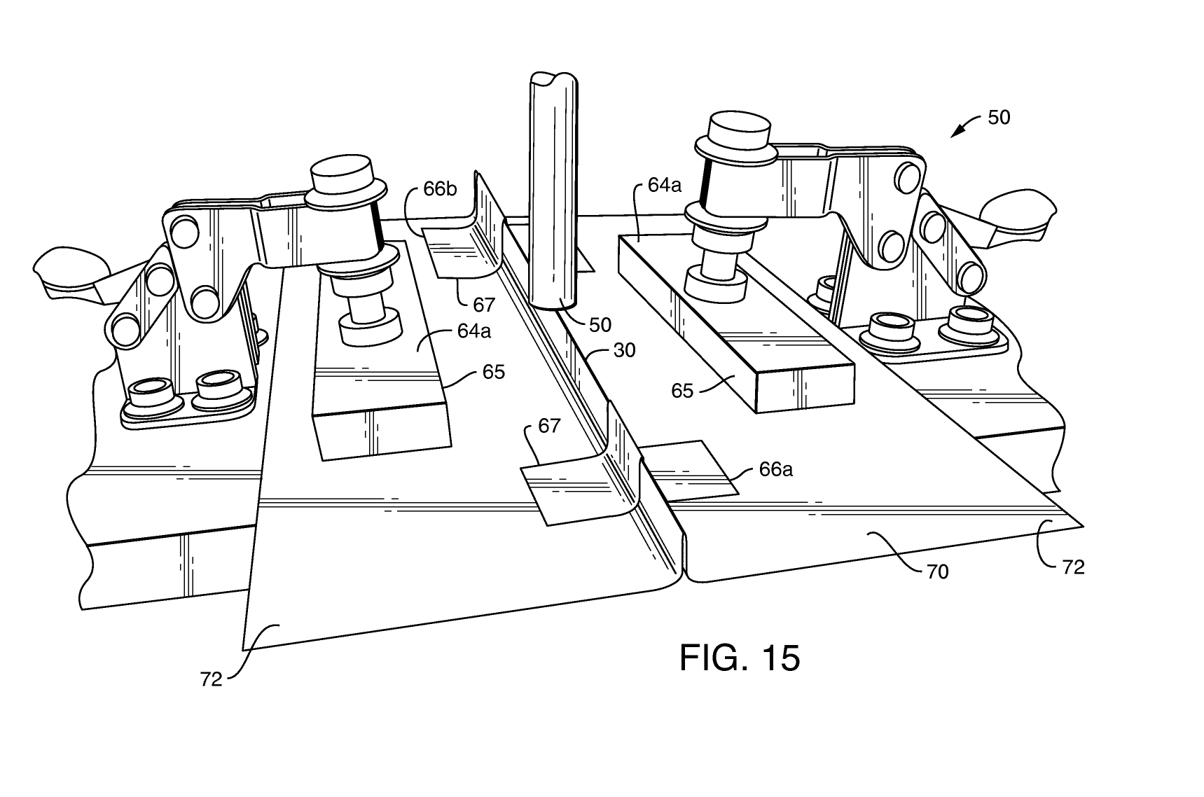

8. Once clamped to the lower mounting fixture, if the fin seam is not substantially vertical (such as if the fin seam is curled over to one side) in the region of the fin seam having close proximity to the path of the cylinder platen, position a first piece of masking tape 66a and a second piece of masking tape 66b such that each extends from one side portion 72 to the opposite side portion 72, each piece of masking tape thereby passing over respective portions of the fin seam 30. The inner edge 67 of the first piece of masking tape is spaced 5 centimeters from the inner edge 67 of the second piece of masking tape (FIGS. 15 and 16). Adjust the attachment positions of the first and second pieces of masking tape in the side portions 72, 72 of the sample so that the fin seam 30 is as much as possible urged into a substantially upright, non-folded-over orientation in the region of the seam 30 between the inner edges 67, 67 of the masking tape pieces having close proximity to the downward path of the cylinder platen.

9. Adjust the position of the sample 70 and sample mounting plate 54 so that the fin seam 30 is directly under the center (+/−2 mm) of the central axis of the cylinder platen 52, and, if masking tape is used to urge the seam upright, so that the central axis of the cylinder platen is equidistant from the inner edges 67, 67 of the first and second pieces of masking tape 66a, 66b (in the direction extending from the front to the back of the compression tester 50) (FIGS. 15 and 16).

10. Activate the downward movement of the platen.

11. The height of each sample is the vertical displacement of the platen (using the zero point determined in step 3 above) when the compression force reaches 0.5 grams.

12. The platen continues to advance down to compress the fin seam. The vertical displacement of the platen (using the zero point determined in step 3 above) at the point where the compression force reaches 2.5 grams is recorded. When the compressive force reaches ten (10) grams, the probe stops and retracts to its starting position.

13. After the cylinder platen has completed its downward and return cycle, the force (grams) and distance (millimeters) data is recorded in the computer and exported to a readable format such as a Windows Excel spreadsheet according to the software operation manual.

14. The rigidity of each sample is the average slope of the compression curve (force versus %-compression) from the point at which the compression force registers 0.5 grams to the point at which the compression force registers 2.5 grams. The equation used to calculate rigidity is given by:

Rigidity=2 grams/[Height(0.5 grams)−Height(at 2.5 grams))/Height(0.5 grams)*100%]
where Height(X) is the height of the seam under force X grams. Height units are in millimeters. The Rigidity is expressed in units of grams-force per %-compression. The difference between Height (0.5 grams) and Height (2.5 grams) can be determined by examining the curves.

15. Test a total of at least sixteen (16) fin seam samples in accordance with steps 5-14 above. For pant-like garments that have left and right fin side seams, this requires a minimum of eight (8) garments. As used herein, “Height” means the average of the height measured on a minimum of sixteen (16) randomly selected fin seam samples. As used herein, “Rigidity” means the average of the rigidity measured on a minimum of sixteen (16) randomly selected fin seam samples.

### Comparative Examples

The height and rigidity of the fin side seams of a variety of disposable absorbent garments available for purchase in the United States were examined.

Description of Products Examined

### TENA® WOMEN™ Heavy Protective Underwear Super Plus Absorbency (“TENA SPA”)

### TENA® Protective Underwear Plus Absorbency (“TENA PA”)

### ASSURANCE® Underwear for Women Extra Absorbency (“ASSURANCE EA”)

### ASSURANCE® Underwear for Women Maximum Absorbency (“ASSURANCE MA”)

### DEPEND® for Women Underwear (“DEPEND”)

### GOODNITES® Underpants (“GOODNITES”)

### POISE® Panty

MOONY PANTS Diaper Pant (Japan; available in U.S. via internet only)

### Experimental Code R

### Experimental Code S

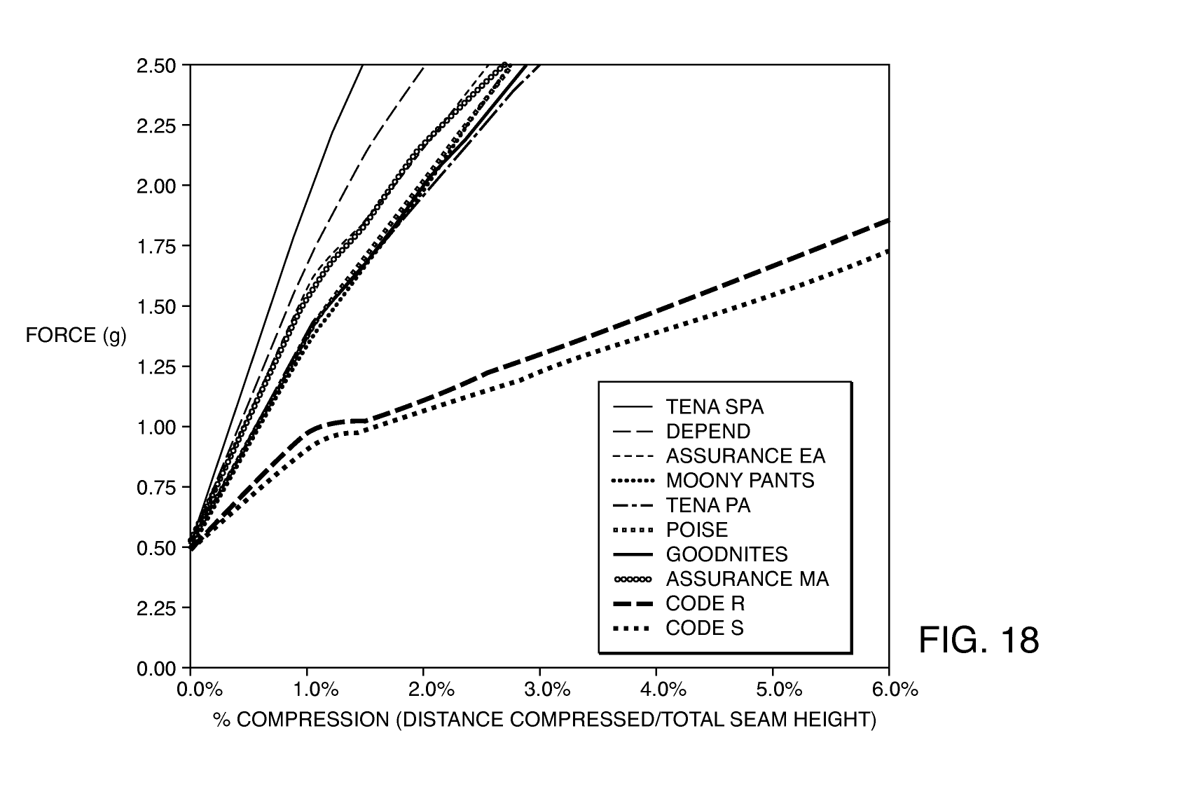

Both Code R and Code S were created by using an elastomeric film laminate material disclosed in U.S. Pat. No. 7,803,244 issued Sep. 28, 2010 to Siqueira et al. and assigned to Kimberly-Clark Worldwide, Inc. to construct the side panels and the center panel of the garment. A front panel web and a back panel web of the material were each stretched to 100% elongation at a tension load of approximately 158 Newtons/meter. While held at that elongation, side seam bonds approximately four (4) millimeters wide were made using an ultrasonic horn and anvil (comprising three columns of dots in the example of FIG. 6). The anvil exhibited the bond pattern shown in FIG. 18. The continuous webs were then cut to leave narrow, unbonded strips extending outwardly from each side seam bond by approximately two (2) millimeters (as measured in the 100% elongated condition). Experimental Code R was first made available for sale in the United States in March of 2012, under the trade name DEPEND REAL FIT for Men briefs. Experimental Code S was first made available for sale in the United States in March of 2012, under the trade name DEPEND SILHOUETTE for Women briefs. Both are manufactured by Kimberly-Clark Global Sales, LLC.

Tables 2 and 3 present the data collected for each product. In each table, the first column lists the product code; the second column lists the number of specimens tested for each product code; the third column lists statistical groupings at the 95% confidence level; and the fourth column present the average value of the parameter in question (i.e., rigidity or height). FIG. 18 presents representative average compression curves for each product code. Each curve presented in FIG. 18 constitutes a representative average of several randomly selected individual compression curves yielded from the individual specimens of each product code, intended to visually depict the relative rigidity (slope) distinctions among the various product codes.

TABLE 2
Comparison of Rigidity

| Product Code | n | Groupings | Avg. Rigidity (Slope) |
|---|---|---|---|
| TENA SPA | 16 | A | 1.340 |
| DEPEND | 16 | B | 0.913 |
| ASSURANCE EA | 16 | B | 0.761 |
| MOONY Pants | 16 | B | 0.729 |
| TENA PA | 16 | B | 0.717 |
| ASSURANCE MA | 16 | B | 0.705 |
| POISE Panty | 18 | B | 0.672 |
| GOODNITES | 6 | BC | 0.656 |
| Experimental Code R | 16 | C | 0.216 |
| Experimental Code S | 16 | C | 0.211 |

TABLE 3
Comparison of Height

| Product Code | n | Groupings | Avg. Height |
|---|---|---|---|
| GOODNITES | 6 | AB | 12.48 |
| TENA PA | 16 | A | 12.24 |
| TENA SPA | 16 | AB | 11.84 |
| ASSURANCE MA | 16 | B | 10.88 |
| ASSURANCE EA | 16 | C | 9.31 |
| DEPEND | 16 | CD | 8.93 |
| POISE Panty | 18 | DE | 7.98 |
| MOONY Pants | 16 | E | 6.77 |
| Experimental Code S | 16 | F | 5.24 |
| Experimental Code R | 16 | F | 5.02 |

As shown by the data in Tables 2 and 3, only the inventors' experimental codes included fin side seams that were both relatively short (e.g., no taller than 6 mm) and relatively soft (e.g., a rigidity no greater than 0.6 grams-force/%-compression). As discussed earlier, it is the combination of these two features in a garment fin seam that can help deliver optimum discretion, comfort, and softness. Due in part to the material, manufacturing, and product integrity challenges associated with creating such garments, prior commercial products have been unable to achieve the combined characteristics of particular embodiments of the present invention.

What is claimed is:

1. A disposable garment having a fin seam, wherein the fin seam has a Height no greater than 6 millimeters and a Rigidity no greater than 0.6 gram-force/%-compression as measured by the Test Method.

2. The disposable garment of claim 1, wherein the fin seam has a Height no greater than 6 millimeters and a Rigidity no greater than 0.5 gram-force/%-compression as measured by the Test Method.

3. The disposable garment of claim 1, wherein the fin seam has a Height no greater than 6 millimeters and a Rigidity no greater than 0.4 gram-force/%-compression as measured by the Test Method.

4. The disposable garment of claim 1, wherein the fin seam has a Height no greater than 5.5 millimeters and a Rigidity no greater than 0.3 gram-force/%-compression as measured by the Test Method.

5. A disposable, pant-like garment having a waist opening, first and second leg openings, a first side fin seam extending substantially from the waist opening to the first leg opening, and a second side fin seam extending substantially from the waist opening to the second leg opening, wherein each side fin seam has a Height no greater than 6 millimeters and a Rigidity no greater than 0.6 gram-force/%-compression as measured by the Test Method.

6. The disposable garment of claim 5, wherein a first front side panel is bonded to a first back side panel along the first side fin seam, and wherein a second front side panel is bonded to a second back side panel along the second side fin seam,
wherein each fin seam is formed via ultrasonic, heat, or pressure bonds, and wherein each side panel comprises an elastomeric film laminate.

7. The disposable garment of claim 5, where both of the side seams extend outward.

8. The disposable garment of claim 7, wherein each fin seam has a Height no greater than 6 millimeters and a Rigidity no greater than 0.5 gram-force/%-compression as measured by the Test Method.

9. The disposable garment of claim 7, wherein each fin seam has a Height no greater than 6 millimeters and a Rigidity no greater than 0.4 gram-force/%-compression as measured by the Test Method.

10. The disposable garment of claim 7, wherein each fin seam has a Height no greater than 5.5 millimeters and a Rigidity no greater than 0.3 gram-force/%-compression as measured by the Test Method.

11. A disposable, pant-like, absorbent garment defining a longitudinal direction and a transverse direction, a front region, a back region, and a crotch region, the garment having a front center panel and a back center panel, the front region including first and second elastomeric front side panels extending transversely from the front center panel, the back region including first and second elastomeric back side panels extending transversely from the back center panel,
wherein the first elastomeric front side panel is bonded to the first elastomeric back side panel along an outwardly extending first side fin seam, and wherein the second elastomeric front side panel is bonded to the second elastomeric back side panel along an outwardly extending second side fin seam,
wherein each side panel comprises an elastomeric film laminate,
wherein each side fin seam has a Height no greater than 6 millimeters and a Rigidity no greater than 0.6 gram-force/%-compression as measured by the Test Method,
wherein the garment comprises an absorbent composite.

12. The absorbent garment of claim 11, wherein the front region further includes an elastomeric front center panel comprising an elastomeric film laminate, wherein the front center panel, the first front side panel, and the second front side panel together integrally form a unitary front body panel,
and wherein the back region further includes an elastomeric back center panel comprising an elastomeric film laminate, wherein the back center panel, the first back side panel, and the second back side panel together integrally form a unitary back body panel.

13. The disposable garment of claim 12, wherein each fin seam has a Height no greater than 6 millimeters and a Rigidity no greater than 0.5 gram-force/%-compression as measured by the Test Method.

14. The disposable garment of claim 12, wherein each fin seam has a Height no greater than 6 millimeters and a Rigidity no greater than 0.4 gram-force/%-compression as measured by the Test Method.

15. The disposable garment of claim 12 wherein each fin seam has a Height no greater than 5.5 millimeters and a Rigidity no greater than 0.3 gram-force/%-compression as measured by the Test Method.
